(19) United States
(12) Patent Application Publication
(10) Pub. No.: US 2011/0092741 A1
(43) Pub. Date: Apr. 21, 2011

(54) PROCESS FOR PREPARING A 1,2-ETHYLENEDIAMINE OR 1,2-PROPYLENEDIAMINE

(76) Inventors: Michael J. Fazio, Midland, MI (US); Erich J. Molitor, Midland, MI (US)

(21) Appl. No.: 12/850,894

(22) Filed: Aug. 5, 2010

Related U.S. Application Data
(60) Provisional application No. 61/252,847, filed on Oct. 19, 2009.

(51) Int. Cl. C07C 209/16

(52) U.S. Cl. 564/480; 564/479

(57) ABSTRACT

The present invention generally relates to a process for preparing a 1,2-ethylenediamine; 1,2-propylenediamine; or a mixture thereof by way of a catalyzed reductive amination reaction employing hydrogen, a reductive amination catalyst, (C₃-C₄₀)polyhydric alcohol having at least three hydroxy groups on consecutive carbon atoms thereof, and primary amine, the catalyzed reductive amination reaction employing reaction conditions that are effective for reductively aminating at least two of the hydroxy groups of the (C₃-C₄₀)polyhydric alcohol and reductively eliminating at least one other of the hydroxy groups of the (C₃-C₄₀)polyhydric alcohol in such a way so as to give a 1,2-ethylenediamine; 1,2-propylenediamine; or a mixture thereof.

### BACKGROUND OF THE INVENTION

1. Field of the Invention

The present invention generally relates to a process for preparing 1,2-ethylenediamines and 1,2-propylenediamines.

2. Description of the Related Art

Compounds known generically as 1,2-ethylenediamines and 1,2-propylenediamines are useful, among other things, as products in chemical industry and starting materials in allied industries such as the polymer, pharmaceutical, biotechnology, and plant and insect control industries. For example, these allied industries can use the 1,2-ethylenediamines and 1,2-propylenediamines as starting monomers in preparations of polymers (e.g., polyamides, polyurethanes, and polyesteramides) and as starting materials in syntheses or preparations of drugs, therapeutic biologics, herbicides, and insecticides. The 1,2-ethylenediamines and 1,2-propylenediamines are also useful in the polymer industry as polymer additives and cross-linking agents for amine-cross-linkable polymers.

U.S. Pat. No. 5,015,773 mentions, among other things, reductive amination of hydroxy-containing compounds such as, among others, polyhydric alcohols (e.g., glycerol) to give an amine product. The amine product corresponds to the hydroxy-containing compound except that at least one hydroxy group (i.e., —OH) of the hydroxy-containing compound has been replaced by either —NH₂ (in reductive aminations with ammonia), —N(H)(aliphatic or aromatic group) (in reductive aminations with a primary amine), or N(aliphatic or aromatic group)₂ (in reductive aminations with a secondary amine). Any other of the hydroxy groups of the hydroxy-containing compound remain in the amine product.

U.S. Pat. No. 5,952,529 mentions, among other things, a catalyst and process for producing amine products from a starting material. The amine product corresponds to the starting material except where at least one amine group (e.g., —NH₂) of the amine product replaces at least one non-amine functional group (e.g., —OH) of the starting material. As in U.S. Pat. No. 5,015,773, any other of the hydroxy groups of starting material remain in the amine product.

Chemical and the allied industries desire an improved process for preparing 1,2-ethylenediamines and 1,2-propylenediamines. Preferably, the process would effectively prepare the 1,2-ethylenediamines and 1,2-propylenediamines directly from polyhydric alcohols and amines. Preferably, the polyhydric alcohols can be obtained from sustainable sources.

### BRIEF SUMMARY OF THE INVENTION

The present invention relates to a process for preparing a 1,2-ethylenediamine; 1,2-propylenediamine; or a mixture of the 1,2-ethylenediamine and 1,2-propylenediamine. In a first embodiment, the present invention is a process for preparing a 1,2-ethylenediamine; 1,2-propylenediamine; or a mixture of the 1,2-ethylenediamine and 1,2-propylenediamine, the process comprising contacting together hydrogen; a reductive amination catalyst; a primary amine; and a (C₃-C₄₀)polyhydric alcohol, the (C₃-C₄₀)polyhydric alcohol having at least three hydroxy groups on consecutive carbon atoms thereof, the contacting being performed under reaction conditions that are effective for reductively aminating at least two of the hydroxy groups of the (C₃-C₄₀)polyhydric alcohol and reductively eliminating at least one other of the hydroxy groups of the (C₃-C₄₀)polyhydric alcohol in such a way so as to give a 1,2-ethylenediamine; 1,2-propylenediamine; or a mixture thereof.

The process of the first embodiment (i.e., invention process) is useful for preparing 1,2-ethylenediamines; 1,2-propylenediamines; and mixtures thereof. The 1,2-ethylenediamines; 1,2-propylenediamines; and mixtures thereof prepared by the invention process are useful as starting monomers in preparations of polymers (e.g., polyamides, polyurethanes, and polyesteramides) and as starting materials in syntheses or preparations of drugs, therapeutic biologics, herbicides, and insecticides. The 1,2-ethylenediamines and 1,2-propylenediamines prepared by the invention process are also useful in the polymer industry as polymer additives and cross-linking agents for amine-cross-linkable polymers (e.g., polymer chains having free —COOH or —CNO groups can be cross-linked to each other by the 1,2-ethylenediamines and 1,2-propylenediamines).

Additional embodiments are described in the remainder of the specification, including the claims.

### DETAILED DESCRIPTION OF THE INVENTION

The present invention relates to the process for preparing a 1,2-ethylenediamine; 1,2-propylenediamine; or a mixture thereof, as summarized previously. In some embodiments, the process prepares the mixture of the 1,2-ethylenediamine; 1,2-propylenediamine. Preferably, the invention process comprises a first step comprising preparing the mixture of 1,2-ethylenediamine and 1,2-propylenediamine as described previously; and a second step comprising purifying at least some of the 1,2-ethylenediamine and 1,2-propylenediamine to give at least one of the 1,2-ethylenediamine and 1,2-propylenediamine in substantially pure form (i.e., 95 weight percent or greater). In some embodiments, the substantially pure 1,2-ethylenediamine or 1,2-propylenediamine can be used directly (i.e., without isolation) in a manufacturing process step as a starting monomer in a preparation of a polymer (e.g., polyamide, polyurethane, or polyesteramide); as a starting material in a synthesis or preparation of a drug, therapeutic biologic, herbicide, or insecticide; or as a polymer additive or cross-linking agent in a process for modulating a property of a polymer or cross-linking an amine-cross-linkable polymer. More preferably, the separating step comprises distilling the mixture. In some embodiments, the process further comprises isolating the substantially pure 1,2-ethylenediamine or 1,2-propylenediamine to give at least one, and even more preferably each of the 1,2-ethylenediamine and 1,2-propylenediamine in substantially pure and isolated (e.g., collected in a container) form.

For purposes of United States patent practice and other patent practices allowing incorporation of subject matter by reference, the entire contents—unless otherwise indicated—of each U.S. patent, U.S. patent application, U.S. patent application publication, PCT international patent application and WO publication equivalent thereof, referenced in the instant Summary or Detailed Description of the Invention are hereby incorporated by reference. In an event where there is a conflict between what is written in the present specification and what is written in a patent, patent application, or patent application publication, or a portion thereof that is incorporated by reference, what is written in the present specification controls.

In the present application, any lower limit of a range of numbers, or any preferred lower limit of the range, may be combined with any upper limit of the range, or any preferred upper limit of the range, to define a preferred aspect or embodiment of the range. Each range of numbers includes all numbers, both rational and irrational numbers, subsumed within that range (e.g., the range from about 1 to about 5 includes, for example, 1, 1.5, 2, 2.75, 3, 3.80, 4, and 5).

In an event where there is a conflict between a unit value that is recited without parentheses, e.g., 2 inches, and a corresponding unit value that is parenthetically recited, e.g., (5 centimeters), the unit value recited without parentheses controls.

As used herein, “a,” “an,” “the,” “at least one,” and “one or more” are used interchangeably. In any aspect or embodiment of the instant invention described herein, the term “about” in a phrase referring to a numerical value may be deleted from the phrase to give another aspect or embodiment of the instant invention. In the former aspects or embodiments employing the term “about,” meaning of “about” can be construed from context of its use. Preferably “about” means from 90 percent to 100 percent of the numerical value, from 100 percent to 110 percent of the numerical value, or from 90 percent to 110 percent of the numerical value. In any aspect or embodiment of the instant invention described herein, the open-ended terms “comprising,” “comprises,” and the like (which are synonymous with “including,” “having,” and “characterized by”) may be replaced by the respective partially closed phrases “consisting essentially of,” consists essentially of,” and the like or the respective closed phrases “consisting of,” “consists of,” and the like to give another aspect or embodiment of the instant invention. In the present application, when referring to a preceding list of elements (e.g., ingredients), the phrases “mixture thereof,” “combination thereof,” and the like mean any two or more, including all, of the listed elements. The term “or” used in a listing of members, unless stated otherwise, refers to the listed members individually as well as in any combination, and supports additional embodiments reciting any one of the individual members (e.g., in an embodiment reciting the phrase “10 percent or more,” the “or” supports another embodiment reciting “10 percent” and still another embodiment reciting “more than 10 percent.”). The term “plurality” means two or more, wherein each plurality is independently selected unless indicated otherwise. The symbols “≦” and “≧” respectively mean less than or equal to and greater than or equal to. The symbols “<” and “>” respectively mean less than and greater than.

The term “(C₃-C₄₀)polyhydric alcohol” means a saturated hydrocarbon of from 3 to 40 carbon atoms, the saturated hydrocarbon being straight or branched and substituted with 3 or more hydroxy groups (i.e., —OH, also known as hydroxyl), each hydroxy group being on a different one of the carbon atoms, at least three of the hydroxy groups being on consecutive carbon atoms, at least one of the at least three hydroxy groups being a primary hydroxy (i.e., an —OH being bonded to a CH₂ group of the saturated hydrocarbon so as to form —CH₂OH). Preferably, the (C₃-C₄₀)polyhydric alcohol means a compound of formula (I):

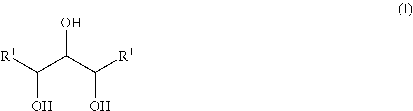

wherein each R¹ independently is H; unsubstituted (C₁-C₃₇)alkyl; or (C₁-C₃₇)alkyl substituted with from 1 to 6 —OH; the total number of carbon atoms of both R¹ being from 0 to 37 carbon atoms; and when the substituted (C₁-C₃₇)alkyl is a substituted (C₁-C₆)alkyl, the number of the —OH of the substituted (C₁-C₆)alkyl being equal to or less than the number of carbon atoms of the substituted (C₁-C₆)alkyl. More preferably, each R¹ is H; or one R¹ is H and the other R¹ is unsubstituted (C₁-C₃₇)alkyl. Still more preferably, the (C₃-C₄₀)polyhydric alcohol is glycerol (i.e., each R¹ is H). Glycerol, also known as glycerin and glycerine, is the compound of the following formula: HOCH₂CH(OH)CH₂OH. Glycerol can be obtained from sustainable sources (e.g., triglycerides).

The term “primary amine” means a compound that is capable of reacting with the (C₃-C₄₀)polyhydric alcohol under the reaction conditions to yield the 1,2-ethylenediamine; 1,2-propylenediamine; or mixture thereof. Preferably, the primary amine is a compound of formula (A):

H₂N—R (A),

wherein R is (C₁-C₄₀)hydrocarbyl or (C₁-C₄₀)heterohydrocarbyl. In some embodiments, R is (C₁-C₄₀)heterohydrocarbyl. In some embodiments, R is (C₁-C₄₀)hydrocarbyl. In some embodiments, R is (C₃-C₄₀)cycloalkyl. In some embodiments, R is (C₁-C₄₀)alkyl. More preferably, R is (C₁-C₁₀)alkyl, and still more preferably (C₁-C₆)alkyl. In some embodiments, the invention process contemplates initially employing the primary amine as a salt (e.g., of a protic acid), and adding a suitable base (e.g., sodium hydride) to a reaction mixture containing the salt in order to generate the free primary amine in situ.

In some embodiments, the present invention also is a 1,2-ethylenediamine; 1,2-propylenediamine; or a mixture thereof prepared by the process of the first embodiment. Preferably, the 1,2-ethylenediamine; 1,2-propylenediamine; or mixture thereof is prepared by the invention process and further purified and isolated in substantially pure form (i.e., 95 weight percent or greater). The terms “1,2-ethylenediamine” and “1,2-propylenediamine” mean a compound of formula (E):

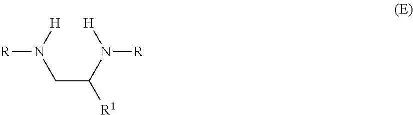

wherein R is (C₁-C₄₀)hydrocarbyl or (C₁-C₄₀)heterohydrocarbyl; and for 1,2-ethylenediamine R¹ is H; and for 1,2-propylenediamine R¹ is —CH₃, —CH₂-(unsubstituted (C₁-C₃₇)alkyl), or —CH₂—((C₁-C₃₇)alkyl substituted with from 1 to 6 —OH as described previously). More preferably, R¹ is H, —CH₃, or —CH₂-(unsubstituted (C₁-C₄)alkyl). Still more preferably, R¹ is H or —CH₃.

In embodiments where the (C₃-C₄₀)polyhydric alcohol is glycerol (i.e., each R¹ is H in formula (I)), the 1,2-ethylenediamine and 1,2-propylenediamine respectively mean a compound of formula (E) wherein R¹ is H or —CH₃, as respectively shown below in formulas (E1) and (E2):

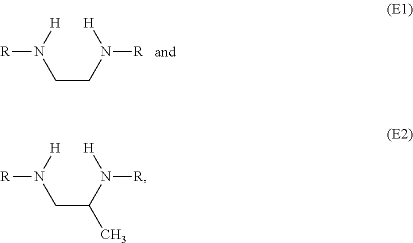

wherein R is as defined previously for formula (E).

The phrase “reductively aminating at least two of the hydroxy groups of the (C₃-C₄₀)polyhydric alcohol” means replacing each of the two —OH groups shown in formula (I) with a group of formula —N(H)R, where R is as defined for the primary amine of formula (A).

The phrase “reductively eliminating at least one other of the hydroxy groups of the (C₃-C₄₀)polyhydric alcohol” means replacing at least one —OH group, or the —CH₂OH group shown in formula (I) with a hydrogen atom.

The term “reductive amination catalyst” means a substance capable of catalyzing the aforementioned reductive amination of the at least two hydroxy groups and, preferably, also catalyzing the aforementioned reductive elimination of the at least one other of the hydroxy groups of the (C₃-C₄₀)polyhydric alcohol. Preferred reductive amination catalysts are those already known in the art for being useful in reductive aminations of alcohols. Examples of such known reductive amination catalysts are BASF Co-0138; Raney cobalt; Raney nickel; Johnson Matthey Co-40-55; and Engelhard/BASF Cu—Co—ZrO₂, which consists essentially of copper (Chemical Abstracts Service (CAS) Registry Number (RegNo.) [7440-50-8], 50 wt %; cobalt (CAS RegNo. [7440-48-4], 45 wt %); and zirconium dioxide (CAS RegNo. [7440-67-7], 5 wt %. In some embodiments, the reductive amination catalyst comprises any one of the catalysts described in U.S. Pat. No. 5,852,529, at column 4, line 37; to column 6, line 62. Still more preferably, the reductive amination catalyst is a (Co, Cu, Ni, Re, and B)-containing catalyst, and even more preferably a (Co, Cu, Ni, Re, and B)-containing catalyst on alumina, which consists essentially of an admixture of gamma alumina (64 wt %); copper (CAS RegNo. [7440-50-8], 5 wt %); cobalt (CAS RegNo. [7440-48-4], 5 wt %); nickel (CAS RegNo. [7440-02-0], 17 wt %); rhenium (5 wt %); and boron (3.7 wt %). Preferably, the reductive amination catalyst is employed in pellet form in a fixed bed reactor so as to facilitate separation of the reductive amination catalyst from the 1,2-ethylenediamine; 1,2-propylenediamine; or the mixture thereof.

Also preferably, the reaction conditions of the invention process further comprise conducting the contacting step with the ingredients being under an initial charging pressure of hydrogen gas of greater than 400 pounds per square inch (psi), i.e., greater than 2800 kilopascals (kPa); and more preferably, 3500 kPa or greater. Preferably, the initial charging pressure of hydrogen gas is 10,000 kPa or lower. The term “initial charging pressure” means the pressure in the reactor just after it is charged with hydrogen gas, which is typically done with the reaction ingredients starting at ambient temperature.

Also preferably, the reaction conditions of the invention process further comprise conducting the contacting step with the ingredients being in contact with each other and are at a reaction temperature of 150 degrees Celsius (° C.) or higher; and more preferably, 160° C. or higher. Preferably, the reaction temperature is 250° C. or lower; more preferably, 200° C. or lower; and still more preferably, 180° C. Typically, heating of the reaction ingredients begins after the initial charging pressure has been achieved as described above.

Preferably, the invention process employs the primary amine and (C₃-C₄₀)polyhydric alcohol in a molar ratio of from about 1.5 to 3 moles of the primary amine per 1 mole of the (C₃-C₄₀)polyhydric alcohol. More preferably, the molar ratio of the primary amine to the (C₃-C₄₀)polyhydric alcohol is from 1.6 to 2.9; still more preferably from 1.8 to 2.4; and even more preferably from 1.9 to 2.1 (e.g., about 2.0).

Even more preferably, the invention process employs the primary amine and (C₃-C₄₀)polyhydric alcohol in a molar ratio of from about 1.5 to 3 moles of the primary amine per 1 mole of the (C₃-C₄₀)polyhydric alcohol; the reaction conditions further comprise the initial charging pressure of hydrogen gas of 3500 kPa or greater; the reaction ingredients are then heated to a reaction temperature of 150° C. or higher; and initial reaction pressure (i.e., pressure upon first reaching the reaction temperature) within the reactor is at least 700 kPa higher than the initial charging pressure.

The invention process is adaptable for use in a batch reactor and process and a continuous flow reactor and process.

Preferably, the compound of formula (E) is prepared as illustrated below in Scheme 1.

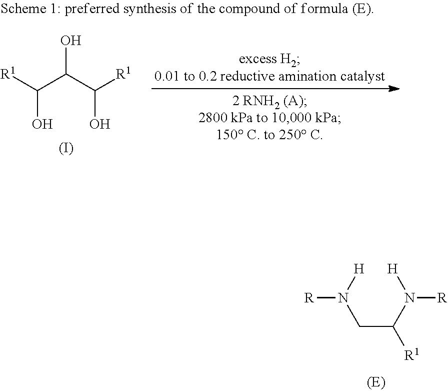

In Scheme 1, load a catalytic amount (e.g., from 0.01 to 0.2 weight of reductive amination catalyst per weight of (C₃-C₄₀)polyhydric alcohol) of one of the aforementioned reductive amination catalysts as a solid into an autoclave reactor (e.g., stainless steel autoclave reactor) under an inert atmosphere (e.g., nitrogen, argon, or helium gas). Then add about two mole equivalents of the primary amine of formula (A) and 1 mole equivalent of the (C₃-C₄₀)polyhydric alcohol of formula (I) to the autoclave reactor, and seal the autoclave reactor and agitate its contents. Charge the autoclave reactor with hydrogen gas to an initial charging pressure of about 500 pounds per square inch gauge (psig; 3500 kPa). Heat the autoclave reactor contents to a heating temperature of from about 150° C. to about 250° C. and an initial reaction pressure. Maintain the heating temperature for a heating period of time of from about 1 hour to about 24 hours. Then allow the reactor contents to cool to room temperature, purge the reactor with the inert gas to remove residual hydrogen gas, and filter the reactor contents (e.g., through a bed of diatomaceous earth) to remove the solid reductive amination catalyst. The resulting filtrate contains one or more reaction products that are compounds of formula (E) and may in some embodiments further contain one or more of unreacted (C₃-C₄₀)polyhydric alcohol of formula (I), unreacted primary amine of formula (A), and by-products (e.g., a by-product of formula HNR₂). Determine yields and relative amounts of the one or more compounds of formula (E) by analyzing the filtrate such as by, for example, gas chromatography (GC) or gas chromatography/mass spectrometry (GC/MS). Optionally employ reference standards of the one or more compounds of formula (E) for quantification of absolute amounts of the one or more compounds of formula (E). In some embodiments, the filtrate containing the one or more compounds of formula (E) (i.e., the 1,2-ethylenediamine, 1,2-propylenediamine, or both) is used directly (that is, without purification) in a subsequent process such as, for example, as a source of one or more monomers in the aforementioned preparation of a polymer (e.g., polyamide, polyurethane, or polyesteramide) or as a source of a starting material in the aforementioned synthesis or preparation of a drug, therapeutic biologic, herbicide, or insecticide; or as the aforementioned polymer additive or cross-linking agent for an amine-cross-linkable polymer.

In other embodiments, the invention process of preparing the one or more compounds of formula (E) further comprises purifying the one or more compounds of formula (E), e.g., the aforementioned filtrate. Preferably, the purifying step comprises acid/base extracting or distilling the one or more compounds of formula (E) from the filtrate so as to separate them from unreacted (C₃-C₄₀)polyhydric alcohol of formula (I) and any non-basic by-products to give a basic first intermediate mixture comprising the compounds of formula (E), any unreacted primary amine of formula (A), and any amine-containing by-products. More preferably, the invention process further comprises purifying the basic first intermediate mixture by distilling the one or more compounds of formula (E) apart from any unreacted primary amine of formula (A), and any amine-containing by-products; or vice versa, distilling any unreacted primary amine of formula (A), and any amine-containing by-products apart from the one or more compounds of formula (E), so as to give in either event a second intermediate mixture wherein at least 90 wt % (preferably at least 95 wt %) thereof consists of the one or more compounds of formula (E) and any remainder is not a compound of formula (E). Still more preferably where the second intermediate mixture contains two or more compounds of formula (E), invention process further comprises purifying the second intermediate mixture containing two or more compounds of formula (E) by distilling the two or more compounds of formula (E) apart from each other, thereby separately providing each one of the two or more compounds of formula (E) independently in a substantially pure form (i.e., greater than 90 wt %, preferably greater than 95 wt %). In some embodiments, each one of the substantially pure compounds of formula (E) can be prepared directly from the filtrate, e.g., by distillation. Preferably, any distilling step is carried out under reduced pressure, that is the distilling step is carried out under less than ambient pressure.

In the reaction illustrated in Scheme 1, reaction conditions such as, for example, heating temperature, heating time, charging pressure of hydrogen gas, or reaction pressure of the reaction for a particular reaction will be adjustable by a person of ordinary skill in the art without undue experimentation in order to optimize the particular reaction. Optimized conditions can vary from reaction to reaction depending upon the particular reductive amination catalyst, primary amine of formula (A), and (C₃-C₄₀)polyhydric alcohol of formula (I) being employed therein. Such optimization is characterizable as being a function of one or more result-effective variables such as, for example, relative amounts of reaction ingredients employed; heating temperature; heating time; reaction pressure; or a combination of two or more thereof. Preferably such optimization results in increasing absolute yield, relative yield, or both of a particular compound of formula (E) (e.g., (E1) or (E2) or both); increasing consumption of the primary amine of formula (A), (C₃-C₄₀)polyhydric alcohol of formula (I), or both; decreasing yield of a reaction by-product, if any; decreasing energy expenditure; or a combination of two or more thereof. Decreasing energy expended conducting a particular reaction comprises, for example, decreasing heating temperature; decreasing heating time; decreasing reaction pressure; or a combination of two or more thereof.

In some embodiments, the primary amine, polyhydric alcohol, 1,2-ethylenediamine, and 1,2-propylenediamine are described as comprising a chemical group (e.g., (C₁-C₄₀)hydrocarbyl). The term “(C₁-C₄₀)hydrocarbyl” means a hydrocarbon radical of from 1 to 40 carbon atoms, wherein each hydrocarbon radical independently is aromatic or non-aromatic, saturated, straight chain or branched chain, cyclic (including mono- and poly-cyclic, fused and non-fused polycyclic) or acyclic, or a combination of two or more thereof; and is unsubstituted. The term “(C₁-C₄₀)heterohydrocarbyl” means a heterohydrocarbon radical of from 1 to 40 carbon atoms and one or more heteroatoms, each heteroatom independently being O; S; S(O); S(O)₂; —NH₂; or N(R^{N}), wherein each R^{N} is H or unsubstituted (C₁-C₁₈)hydrocarbyl. The heterohydrocarbon radical is on a carbon atom or nitrogen atom thereof. Each heterohydrocarbon radical independently is unsubstituted, aromatic or non-aromatic, saturated, straight chain or branched chain, cyclic (including mono- and poly-cyclic, fused and non-fused polycyclic) or acyclic, or a combination of two or more thereof. Any atom of a chemical group that is not specified herein is understood to be a hydrogen atom. Preferably, there are no O—O, S—S, or O—S bonds, other than O—S bonds in an S(O) or S(O)₂ diradical functional group, in (C₁-C₄₀)hydrocarbyl and (C₁-C₄₀)heterohydrocarbyl.

### Materials and General Methods

### General Description of a Preferred Invention Process

Charge a 300 milliliter (mL) volume autoclave reactor under a nitrogen gas atmosphere with a catalytic amount of 4.0 grams (g) of the solid reductive amination catalyst, Engelhard/BASF Cu—Co—ZrO₂ or a catalytic amount of 2.0 g of the solid reductive amination catalyst that is the aforementioned (Co, Cu, Ni, Re, and B)-containing catalyst on alumina. Then add 80 g (i.e., two mole equivalents) of normal-butyl amine (i.e., CH₃CH₂CH₂CH₂NH₂) and 50 g (i.e., 1 mole equivalent) of glycerine to the reactor. Seal the reactor and agitate its contents while pressurizing the reactor with hydrogen gas to an initial charging pressure of hydrogen gas of 500 psig (3500 kPa). Heat the reactor contents to a heating temperature of either 160° C. or 180° C. as indicated for particular Examples later) and an initial reaction pressure; record the initial reaction pressure; and maintain the heating temperature for a heating period of time of 3 hours. Then allow the reactor contents to cool to room temperature, purge the reactor with nitrogen gas to remove residual hydrogen gas, and filter the reactor contents through a bed of pre-wetted diatomaceous earth to remove the solid reductive amination catalyst, optionally rinsing the filtercake with additional 44 g or normal-butyl amine (21 to 22 g of the 44 g of normal-butyl amine is used to prepare the pre-wetted diatomaceous earth by wetting dry diatomaceous earth therewith); and provide a filtrate containing one or more reaction products that are compounds of formula (E). Determine percent conversion of glycerol and yields and relative amounts of the compounds of formula (E) by gas chromatography using a 5% phenyl methyl silicone column (Agilent 19091J-413; HP-5, 30 meters length, 0.32 millimeters inner diameter, 0.25 micron film thickness; Agilent Technologies Inc., Santa Clara, Calif., USA), carrier gas helium flowing at 50 milliliters per minute; injection volume 1 microliter; oven temperature programmed heating rate from 100° C. to 250° C. at 10° C. per minute and a flame ionization detector.

### EXAMPLE(S) OF THE PRESENT INVENTION

Non-limiting examples of the present invention are described below. In some embodiments, the present invention is as described in any one of the examples.

### Examples 1 to 4

Following the general description of a preferred invention process described in the Materials and General Methods, carry out four invention processes using the reaction conditions and amounts listed below in Table 1.

TABLE 1
reaction conditions and amounts of Examples 1 to 4

| | | | | | | Total | | |
|---|---|---|---|---|---|---|---|---|
| | | | | Reaction | | Amt. | | |
| | | Amt. | | Amt. of | Reaction | extra n- | | |
| | | Red. | | starting | Amt. of | BuNH₂ | initial | Weight |
| | | Amin | | n- | starting | used in | reaction | of |
| Ex. | | Cat. | Temp. | BuNH₂ | glycerol | filtering | pressure | filtrate |
| No. | Red. Amin. Cat. | (g) | (° C.) | (g) | (g) | step (g) | (psig) | (g) |
| 1 | (Co, Cu, Ni, Re, | 2.01 | 160 | 80.1 | 50.1 | 44.3 | 688 | 168.6 |
| | and | | | | | | | |
| | B)-containing | | | | | | | |
| | catalyst/alumina | | | | | | | |
| 2 | (Co, Cu, Ni, Re, | 2.02 | 180 | 80.1 | 50.1 | 44.2 | 682 | 168.7 |
| | and | | | | | | | |
| | B)-containing | | | | | | | |
| | catalyst/alumina | | | | | | | |
| 3 | Cu—Co—ZrO₂ | 4.02 | 160 | 80.0 | 50.2 | 0 | 640 | 80.4* |
| 4 | Cu—Co—ZrO₂ | 4.03 | 180 | 80.1 | 50.0 | 0 | 650 | 57.2* |

“Ex. No.” means Example Number;
“Red. Amin Cat.” means reductive amination catalyst;
“Amt.” means amount;
g means grams;
“Temp.” means temperature;
“Reaction Amt.” means amount used in reaction before filtration;
“n-BuNH₂” means normal-butyl amine;
688 psig = 4820 kPa;
682 psig = 4770 kPa;
640 psig = 4500 kPa;
650 psig = 4600 kPa;
*about 50 g and 70 g of filtrate are respectively remain in diatomaceous earth in Examples 3 and 4 because Examples 3 and 4 do not employ extra n-BuNH₂ to wash remaining filtrate from same.

Analyze the filtrate products of Examples 1 to 4 using the GC method described previously to identify and determine relative amounts (product distribution) of compounds of formula (E) of 1,2-dibutylaminoethane (DBE; n-butyl-N(H)CH₂CH₂N(H)-n-butyl) and 1,2-dibutylaminopropane (DBP; n-butyl-N(H)CH₂CH(CH₃)N(H)-(n-butyl); and by-product dibutylamine (DBA; (n-butyl)₂NH) and determine percent conversion of glycerol to total of the DBE and DBP. Results are reported below in Table 2.

TABLE 2
GC reaction results for Examples 1 to 4

| | Percent | Proportion of By-product DBA | Proportion of product DBE | Proportion of product DBP |
|---|---|---|---|---|
| Ex. | glycerol | (fraction of total of | (fraction of total of | (fraction of total of |
| No. | conversion (%) | DBA + DBE + DBP) | DBA + DBE + DBP) | DBA + DBE + DBP) |
| 1 | 48 | 0.92 | 0.05 | 0.04 |
| 2 | 61 | 0.93 | 0.05 | 0.03 |
| 3 | 40 | 0.78 | 0.11 | 0.11 |
| 4 | 62 | 0.77 | 0.10 | 0.13 |

Isolate and condense the DBE and DBP eluting from the GC column in substantially pure forms thereof to independently give an isolated liquid sample of DBE having a purity of greater than 95 wt % and an isolated liquid sample of DPE having a purity of greater than 95 wt %.

As shown by the Examples, the process of the first embodiment is useful in preparing 1,2-ethylenediamines and 1,2-propylenediamines from (C₃-C₄₀)polyhydric alcohols of formula (I) and primary amines of formula (A). The 1,2-ethylenediamines and 1,2-propylenediamines can be prepared as mixtures thereof and preferably are purified (e.g., by distillation or extraction/distillation) to independently give each 1,2-ethylenediamine and 1,2-propylenediamine in a substantially pure form (e.g., greater than 95 wt % purity).

While the invention has been described above according to its preferred embodiments, it can be modified within the spirit and scope of this disclosure. This application is therefore intended to cover any variations, uses, or adaptations of the instant invention using the general principles disclosed herein. Further, the instant application is intended to cover such departures from the present disclosure as come within the known or customary practice in the art to which this invention pertains and which fall within the limits of the following claims.

1. A process for preparing a 1,2-ethylenediamine, 1,2-propylenediamine, or a mixture of the 1,2-ethylenediamine and 1,2-propylenediamine, the process comprising contacting together hydrogen; a reductive amination catalyst; a primary amine; and a (C₃-C₄₀)polyhydric alcohol, the (C₃-C₄₀)polyhydric alcohol having at least three hydroxy groups on consecutive carbon atoms thereof, the contacting being performed under reaction conditions that are effective for reductively aminating at least two of the hydroxy groups of the (C₃-C₄₀)polyhydric alcohol and reductively eliminating at least one other of the hydroxy groups of the (C₃-C₄₀)polyhydric alcohol in such a way so as to give a 1,2-ethylenediamine; 1,2-propylenediamine; or a mixture thereof.

2. The process as in claim 1, the (C₃-C₄₀)polyhydric alcohol being a compound of formula (I): 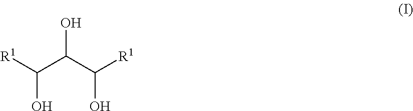
wherein each R¹ independently is H; unsubstituted (C₁-C₃₇)alkyl; or (C₁-C₃₇)alkyl substituted with from 1 to 6 —OH; the total number of carbon atoms of both R¹ being from 0 to 37 carbon atoms; and when the substituted (C₁-C₃₇)alkyl is a substituted (C₁-C₆)alkyl, the number of the —OH of the substituted (C₁-C₆)alkyl being equal to or less than the number of carbon atoms of the substituted (C₁-C₆)alkyl.

3. The process as in claim 2, the (C₃-C₄₀)polyhydric alcohol being glycerol.

4. The process as in claim 1, the primary amine being a compound of formula (A):
H₂N—R (A),
wherein R is (C₁-C₄₀)hydrocarbyl or (C₁-C₄₀)heterohydrocarbyl.

5. The process as in claim 4, wherein R is (C₁-C₄₀)hydrocarbyl.

6. The process as in claim 5, the (C₁-C₄₀)hydrocarbyl being (C₁-C₄₀)alkyl.

7. The process as in claim 1, the reductive amination catalyst comprising BASF Co-0138; Raney cobalt; Raney nickel; Johnson Matthey Co-40-55; Engelhard/BASF Cu—Co—ZrO₂; or a (Co, Cu, Ni, Re, and B)-containing catalyst.

8. The process as in claim 7, the reductive amination catalyst comprising Engelhard/BASF Cu—Co—ZrO₂; or the (Co, Cu, Ni, Re, and B)-containing catalyst.

9. The process as in claim 1, the 1,2-ethylenediamine and 1,2-propylenediamine being a compound of formula (E): 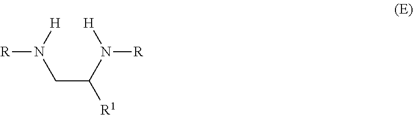
wherein R is (C₁-C₄₀)hydrocarbyl or (C₁-C₄₀)heterohydrocarbyl; and for 1,2-ethylenediamine R¹ is H; and for 1,2-propylenediamine R¹ is —CH₃, —CH₂-(unsubstituted (C₁-C₃₇)alkyl), or —CH₂—((C₁-C₃₇)alkyl substituted with from 1 to 6 —OH).

10. The process as in claim 9, wherein R¹ of formula (E) is H or —CH₃.

11. The process as in claim 1, wherein the (C₃-C₄₀)polyhydric alcohol being glycerol; the primary amine being a compound of formula H₂N—(C₁-C₄₀)alkyl; the reductive amination catalyst comprising Engelhard/BASF Cu—Co—ZrO₂ or a (Co, Cu, Ni, Re, and B)-containing catalyst; and the, 2-ethylenediamine and 1,2-propylenediamine respectively being a compound of formula (E1) and (E2): 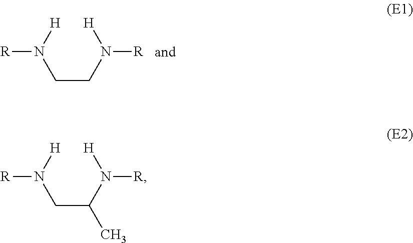
wherein R is —(C₁-C₄₀)alkyl.

12. The process as in claim 1, the process further comprising purifying at least some of the 1,2-ethylenediamine; 1,2-propylenediamine; or both to give at least one of the 1,2-ethylenediamine and 1,2-propylenediamine in substantially pure (i.e., 95 weight percent or greater) form.

13. The process as in claim 12, the purifying comprising distilling.

14. The process as in claim 12, the process further comprising isolating the substantially pure 1,2-ethylenediamine or 1,2-propylenediamine to give at least one of the 1,2-ethylenediamine and 1,2-propylenediamine in substantially pure and isolated form.
